(19) United States
(12) Patent Application Publication
(10) Pub. No.: US 2014/0243691 A1
(43) Pub. Date: Aug. 28, 2014

(54) MEASUREMENT DEVICE, INDEX CALCULATING METHOD, AND INDEX CALCULATING PROGRAM

(71) Applicant: OMRON HEALTHCARE CO., LTD., Muko-shi, Kyoto (JP)

(72) Inventors: Toshiyuki Osaki, Kyoto (JP); Naoki Mori, Kyoto (JP); Toshihiko Ogura, Kyoto (JP); Takashi Honda, Niwa-gun (JP)

(21) Appl. No.: 14/348,473

(22) PCT Filed: Oct. 9, 2012
(86) PCT No.: PCT/JP2012/076111

§ 371 Date: Mar. 28, 2014

(30) Foreign Application Priority Data
Oct. 28, 2011 (JP) 2011-237574

(51) Int. Cl. A61B 5/021; A61B 5/00

(52) U.S. Cl. CPC A61B 5/02108; A61B 5/6802; A61B 5/7235; A61B 5/742
USPC 600/490; 600/485

(57) ABSTRACT

A measurement device includes a pulse wave measurement unit for measuring a pulse wave, and a calculation unit for calculating a predetermined parameter value from the pulse wave, and calculating an index value corresponding to Ankle Brachial Blood Pressure Index as an index value of arteriostenosis using the parameter value.

### TECHNICAL FIELD

The present invention relates to a measurement device, an index calculating method, and an index calculating program. Specifically, the present invention relates to a measurement device for measuring a biological value to calculate an index value related to angiostenosis, and a method and a program for calculating the index.

### BACKGROUND ART

Conventionally, Ankle Brachial Blood Pressure Index (ABI), which is the ratio of blood pressures in the lower and upper limbs, is used as an index of angiostenosis.

For example, as disclosed in JP 2004-261319A, the ABI has been obtained by measuring the blood pressures in the lower and upper limbs of a subject in the supine position with a blood pressure measurement device and then calculating the ratio of these pressures.

### CITATION LIST

### Patent Literature

Patent Literature 1: JP 2004-261319A

### SUMMARY OF INVENTION

### Technical Problem

However, if the subject suffers from severe arterial calcification it may not be possible to accurately measure blood pressures due to insufficient compression of the arteries. Therefore, in this case, there is a problem that accuracy of ABI as an index of angiostenosis is decreased.

Furthermore, if the subject is afflicted with unstable pulse amplitude due to arrhythmia or small pulse amplitude due to angiostenosis, it may not be possible to accurately measure blood pressures. Therefore, in this case, there is also a problem that accuracy of ABI as an index of angiostenosis is decreased.

Moreover, there are cases where some subjects suffer from pain due to the measurement because a blood pressure measurement in the upper limbs and lower limbs is required in order to calculate ABI as described above, and therefore there is also a problem that the measurement is sometimes a great burden on the subjects. There is also a problem that measurement time for the blood pressure measurement is required.

Furthermore, as described above, a subject needs to be in the supine position in order to calculate ABI, and therefore there is also a problem that the measurement lacks simplicity as a screening test.

The present invention was made in view of these problems, and it is an object thereof to provide a measurement device for easily and accurately calculating an index value related to angiostenosis while suppressing a burden on subjects, and a method and a program for calculating the index.

### Solution to Problem

In order to achieve the above-described object, in an aspect of the present invention, a measurement device is a measurement device for measuring a pulse wave and calculating an index of arteriostenosis from the pulse wave, and includes a measurement unit to be mounted on a measurement site for measuring a value corresponding to a load given to the measurement site and an arithmetic device connected to the measurement unit. The arithmetic device includes a pulse wave measurement unit for measuring a pulse wave based on a measurement value in the measurement unit, a first calculation unit for calculating a predetermined parameter value from the pulse wave, and a second calculation unit for calculating an index value corresponding to Ankle Brachial Blood Pressure Index (ABI) as the index of arteriostenosis using the parameter value.

Preferably, the measurement unit includes a cuff for being mounted on the measurement site and a sensor for detecting a pressure inside the cuff, the arithmetic device is connected to the sensor, and the pulse wave measurement unit measures a pulse wave from the sensor.

Preferably, the first calculation unit calculates, as the predetermined parameter value, from the pulse wave, at least one of a normalized pulse wave area (% MAP), which is an index indicating a sharpness of the pulse wave, an upstroke time (UT), which is an index indicating a rising feature value of an ankle pulse wave, a pulse amplitude, and an index value indicating a lower limb-upper limb pulse wave transfer function, which is a function for transfer of a pulse wave from the upper limb to the lower limb.

More preferably, the second calculation unit calculates the index value by combining two or more of % MAP, UT pulse amplitude, and the index value indicating a lower limb-upper limb pulse wave transfer function that are calculated by the first calculation unit.

More preferably, the second calculation unit calculates the index value by combining the index value indicating a lower limb-upper limb pulse wave transfer function and at least one of the % MAP, UT, and pulse amplitude that are calculated by the first calculation unit.

In another aspect of the present invention, an index calculating method is a calculating method for calculating an index value of arteriostenosis from a pulse wave, and includes the steps of obtaining the pulse wave, calculating a predetermined parameter value from the pulse wave, and calculating an index value corresponding to Ankle Brachial Blood Pressure Index (ABI) as the index of arteriostenosis using the parameter value.

In yet another aspect of the present invention, an index calculating program is a program for causing a computer to execute processing for calculating an index value of arteriostenosis from a pulse wave, and causes the computer to execute the steps of obtaining the pulse wave, calculating a predetermined parameter value from the pulse wave, and calculating an index value corresponding to Ankle Brachial Blood Pressure Index (ABI) as the index of arteriostenosis using the parameter value.

### Advantageous Effects of Invention

With the present invention, it is possible to calculate, from a pulse wave, an index value corresponding to ABI, which is an angiostenosis-related index value that is conventionally calculated from a blood pressure value.

### BRIEF DESCRIPTION OF DRAWINGS

FIG. 1 shows an exemplary configuration of a measurement device according to an embodiment of the present invention.

FIG. 2 is a block diagram showing a specific example of the functional configuration of the measurement device in FIG. 1.

FIG. 3 is a graph showing a correlation between the ABI and % MAP.

FIG. 4 is a graph showing a correlation between the ABI and UT.

FIG. 5 is a graph showing a correlation between the ABI and pulse amplitude.

FIG. 6 is a graph showing a correlation between the ABI and the EABI, which is an index calculated from pulse waves.

FIG. 7 is a diagram showing detailed measurement results of the subject from whom the measurements denoted by P1 in FIG. 6 were taken.

FIG. 8 is a diagram showing detailed measurement results of the subject from whom the measurements denoted by P2 in FIG. 6 were taken.

FIG. 9 is a diagram showing detailed measurement results of the subject from whom the measurements denoted by P3 in FIG. 6 were taken.

FIG. 10 illustrates graphs that show measurement results of pulse waves in the right ankle (A) and the left ankle (B) of a healthy subject.

FIG. 11 is a graph showing the step response for the right upper arm to the right ankle (the right step response) calculated from the pulse waves measured in the right ankle of FIG. 10(A) and the pulse waves measured in the right upper arms.

FIG. 12 is a graph showing the step response for the left upper arm to the left ankle (the left step response) calculated from the pulse waves measured in the left ankle of FIG. 10(B) and the pulse waves measured in the left upper arms.

FIG. 13 is a graph comparing the right step response of FIG. 11 and the left step response of FIG. 12.

FIG. 14 is an X-ray image showing the arterial condition of a patient with arteriosclerosis obliterans who is a measurement subject.

FIG. 15 illustrates graphs showing measurement results of pulse waves in the right upper arm (A) and the right ankle (B) of the patient shown in FIG. 14.

FIG. 16 illustrates graphs showing measurement results of pulse waves in the left upper arm (A) and the left ankle (B) of the patient shown in FIG. 14.

FIG. 17 illustrates a graph showing the right step response calculated from pulse waves measured in the right upper arm and the right ankle shown in FIG. 15.

FIG. 18 illustrates a graph showing the left step response calculated from pulse waves measured in the left upper arm and the left ankle shown in FIG. 16.

FIG. 19 is a graph comparing the right step response of FIG. 17 with the left step response of FIG. 18.

FIG. 20 is a schematic diagram of the Avolio Model.

FIG. 21 is a table showing the degrees of stenosis created in the segments designated by the element numbers 82, 104, and 111 (circled in FIG. 20) in the Avolio Model that were used by the inventors for performing calculations.

FIG. 22 is a graph plotting the results of the calculations performed by the inventors.

FIG. 23 is a graph describing the upper area, the ratio of the upper area to the lower area, and the maximum value defined in a step response interval.

FIG. 24 is a graph showing a correlation between the ABI and the upper area of the step response.

FIG. 25 is a graph showing a correlation between the ABI and the ratio of the upper area to the lower area of the step response.

FIG. 26 is a graph showing a correlation between the ABI and the maximum value of the step response interval.

FIG. 27 is a graph showing a correlation between the ABI and the EABI.

FIG. 28 is a flowchart representing a specific example of the operational flow that occurs in the measurement device.

FIG. 29 is a flowchart representing a specific example of the operation in Step S113 of FIG. 28.

### DESCRIPTION OF EMBODIMENTS

Preferred embodiments of the present invention will be described hereinafter with specific reference to the attached drawings. The same numerals refer to the same components and elements throughout the description and the drawings, such that the designations and functions of these elements are also identical.

System Configuration

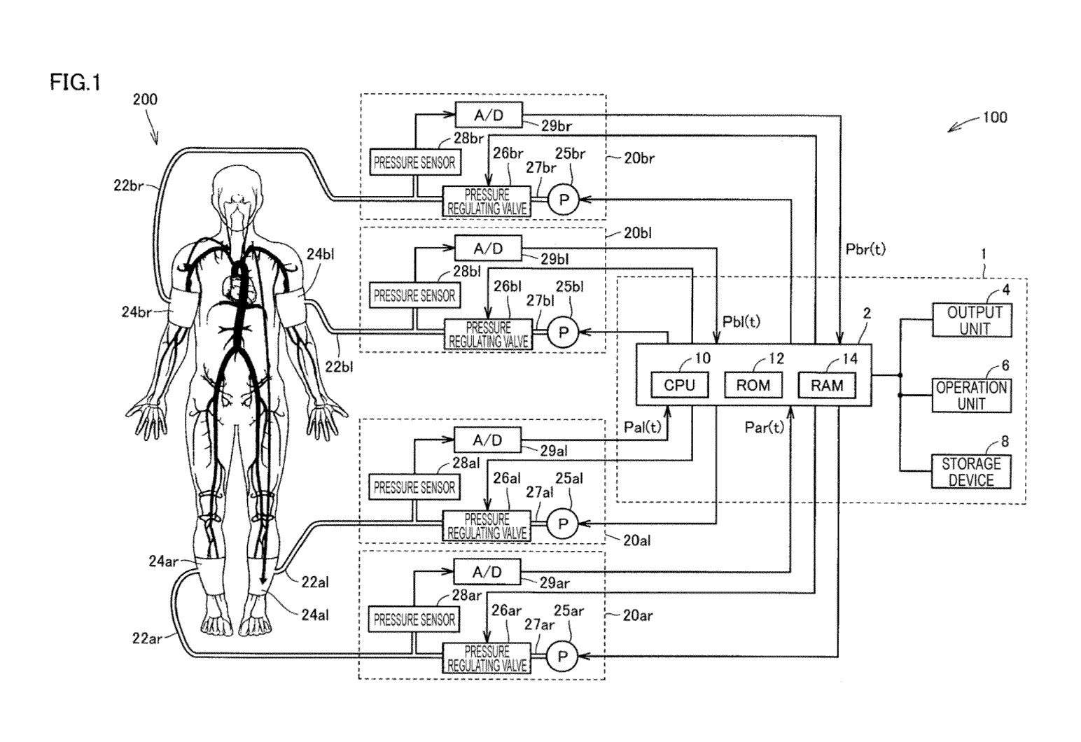

FIG. 1 shows an exemplary configuration of a measurement device 100 according to an embodiment of the present invention.

Referring to FIG. 1, the measurement device 100 includes an information processing unit 1, four detection units 20ar, 20al, 20br, and 20bl, and four cuffs 24ar, 24al, 24br, and 24bl.

The cuffs 24br, 24bl, 24ar, and 24al are worn on respective extremities of a subject 200. Specifically, they are respectively worn on the right upper arm (right upper limb), left upper arm (left upper limb), right ankle (right lower limb), and left ankle (left lower limb). As used herein, the term “extremity” refers to a site on any of the four limbs, and may be a wrist, a fingertip, or the like. Throughout the specification, the cuffs 24ar, 24al, 24br, and 24bl will be collectively referred to as “cuffs 24” unless there is a need to distinguish between individual cuffs.

The detection units 20ar, 20al, 20br, and 20bl each include hardware necessary for detecting pulse waves in an extremity of the subject 200. As all the detection units 20ar, 20al, 20br, and 20bl may have an identical configuration, they will be collectively referred to as “detection units 20” unless there is a need to distinguish between individual units.

The information processing unit 1 includes a control unit 2, an output unit 4, an operation unit 6, and a storage device 8.

The control unit 2 is a device that performs overall control of the measurement device 100 and is typically implemented by a computer that comprises a CIPU (central processing unit) 10, a ROM (read only memory) 12, and a RAM (random access memory) 14.

The CPU 10 corresponds to an arithmetic processing unit, reads a program previously stored in the ROM 12, and executes the program while using the RAM 14 as the work memory.

Additionally, the output unit 4, the operation unit 6, and the storage device 8 are connected to the control unit 2. The output unit 4 outputs measured pulse waves, the result of analysis of pulse waves, and the like. The output unit 4 may be, for example, a display device implemented by LEDs (light emitting diodes) or an LCD (liquid crystal display), or a printer (driver).

The operation unit 6 is adapted to receive instructions from a user. The storage device 8 is adapted to hold various types of data and programs. The CPU 10 of the control unit 2 reads data and programs stored in the storage device 8 as well as performing writing to the storage device 8. For example, the storage device 8 may be implemented by a hard disk drive, nonvolatile memory (e.g., a flash memory), or a removable recording medium.

The specific configuration of each of the detection units 20 is described hereinafter. The detection unit 20br detects pulse waves in the right upper arm by adjusting and detecting the internal pressure of the cuff 24br worn by the subject 200 on the right upper arm (hereinafter “cuff pressure”). The cuff 24br contains a fluid bag (not shown), such as an air bag.

The detection unit 20br includes a pressure sensor 28br, a pressure regulating valve 26br, a pressure pump 25br, an A/D (analog-to-digital) converter 29br, and a tube 27br. The cuff 24br is connected to the pressure sensor 28br and the pressure regulation valve 26br via the tube 22br.

The pressure sensor 28br is a device for detecting pressure fluctuation transmitted through the tube 22br and may be implemented, for example, on a semiconductor chip made of single crystal silicon or any other suitable material. A signal representing the pressure fluctuation detected by the pressure sensor 28br is converted to a digital signal by the A/D converter 29br and sent to the control unit 2 as a pulse wave signals pbr(t).

The pressure regulating valve 26br is interposed between the pressure pump 25br and the cuff 24br and maintains the pressure used for pressurizing the cuff 24br in a predetermined range during measurement. The pressure pump 25br operates in accordance with a detection instruction from the control unit 2 to supply air to the fluid bag (not shown) in the cuff 24br in order to pressurize the cuff 24br.

This pressurization of the fluid bag causes the cuff 24br to press against the measurement site, such that pressure variations corresponding to pulse waves in the right upper arm may be transmitted to the detection unit 20br via the tube 22br. The detection unit 20br detects the pulse waves at the right upper arm by detecting the pressure variations transmitted thereto.

Similarly, the detection unit 20bl includes a pressure sensor 28b, a pressure regulating valve 26bl, a pressure pump 25bl, an A/D converter 29b1, and a tube 27bl. The cuff 24bl is connected to the pressure sensor 28b1 and the pressure regulation valve 26bl by the tube 22b1.

Likewise, the detection unit 20ar includes a pressure sensor 28ar, a pressure regulating valve 26ar, a pressure pump 25ar, an A/D convener 29ar, and a tube 27ar. The cuff 24ar is connected to the pressure sensor 28ar and the pressure regulating valve 26ar via the tube 22ar.

Similarly, the detection unit 20al includes a pressure sensor 28al, a pressure regulating valve 26al, a pressure pump 25al, an A/D converter 29al, and a tube 27al. The cuff 24al is connected to the pressure sensor 28al and the pressure regulating valve 26al via the tube 22al.

As the functions of the components in the detection units 20bl, 20ar, and 20al are identical to those of the detection unit 20br, detailed description thereof is omitted. Likewise, reference symbols, such as “ar” and “br,” are omitted from the description of the components in the detection units 20 hereinafter unless there is a need to distinguish between them.

It should be noted that although a configuration for detecting pulse waves using the pressure sensors 28 is described in this embodiment, it is possible to use a configuration for detecting pulse waves using arterial volume sensors (not shown). In this case, such arterial volume sensors may include a light-emitting device for irradiating an artery and a light-receiving element for receiving the light irradiated by the light-emitting device after it is transmitted through or reflected by the artery. An alternative configuration may include a plurality of electrodes for feeding a minute constant current to the measurement site of the subject 200 so as to detect the voltage variations caused by the variations in impedance (bioelectrical impedance) that occur in accordance with the pulse wave propagation.

Overview of the Operation

In the measurement device 100 of the present embodiment, an index indicating the presence or absence of stenosis or the degree of stenosis in arteries corresponding to Ankle Brachial Blood Pressure Index (ABI), which is the ratio of the blood pressure values measured in the upper and lower limbs, is calculated from pulse waves measured in the upper and lower limbs.

Functional Configuration

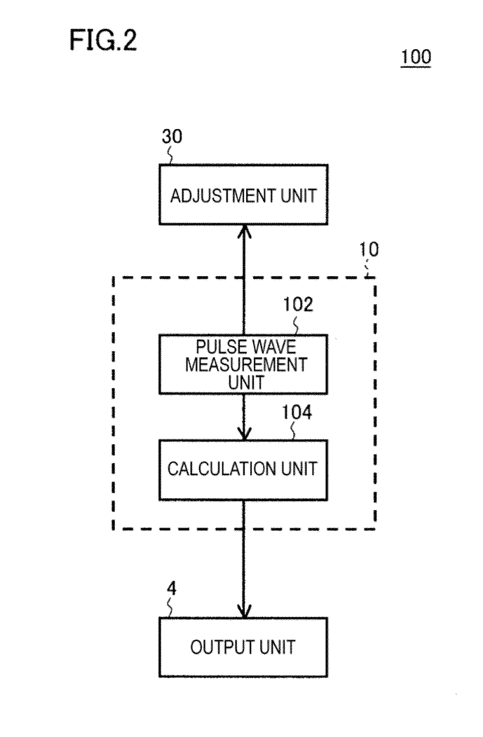

FIG. 2 is a block diagram showing a specific example of the functional configuration of the measurement device 100 for performing the operation as described above.

The functions shown in FIG. 2 are implemented mainly on the CPU 10 as the CPU 10 reads out a program stored in the ROM 12 and executes the program while using the RAM 14 as the work memory. However, at least part of the functions may be implemented by the system configuration shown in FIG. 1 or other hardware, such as electric circuitry.

With reference to FIG. 2, the measurement device has various functions implemented therein, including an adjustment unit 30, a pulse wave measurement unit 102, a calculation unit 104 for calculating the above-described index, and an output unit 4.

The adjustment unit 30 is a functional unit for adjusting the pressure inside the cuffs 24. The functionality of the adjustment unit 30 may be implemented, for example, by the pressure pump 25 and the pressure regulating valve 26 shown in FIG. 1.

The pulse wave measurement unit 102 is connected to the adjustment unit 30 and the A/D converter 29 for performing processing necessary to measure the pulse wave (PVR) in the extremity. The pulse wave measurement unit 102 adjusts the pressure inside the cuff 24 by providing a command signal to the adjustment unit 30 and receives cuff pressure signals Par(t), Pal(t), Pbr(t), and Pbl(t) detected in response to the command signal. Subsequently, pulse waveforms for multiple heartbeats are obtained in each extremity by recording the received cuff pressure signals Par(t), Pal(t), Pbr(t), and Pbl(t) in time series. The pulse wave measurement is performed, for example, for a predetermined duration of time (e.g., approximately 10 seconds).

The following describes an index of arteriostenosis that is calculated from the pulse waves measured in the upper and the lower limbs and corresponds to ABI.

Examples of indices of arteriostenosis using pulse waves include not only pulse amplitude but also an index indicating sharpness of a pulse wave, which is referred to as a normalized pulse wave area (% MAP). % MAP is calculated, for example, as the ratio of M to H (% MAP=M/H×100), where M is the height from the diastolic pressure when the pulse wave area is leveled and H is the peak height of the pulse wave (i.e., pulse pressure). An index value of the % MAP increases in the presence of arteriostenosis or arterial occlusion.

Moreover, another example is an index indicating a rising feature value of an ankle pulse wave, which is referred to as an upstroke time (UT). The UT is calculated as the rising period of the ankle pulse wave from the rising point to the peak. If arteriostenosis or arterial occlusion exists in the subject, the above-described period is extended, thus increasing an index value of the UIT.

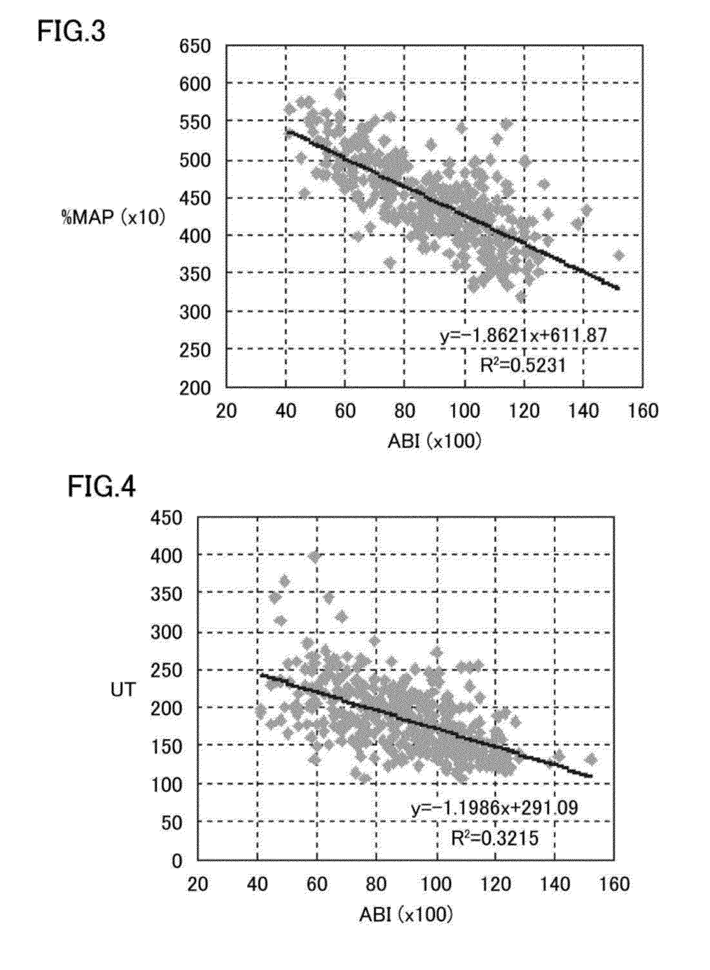

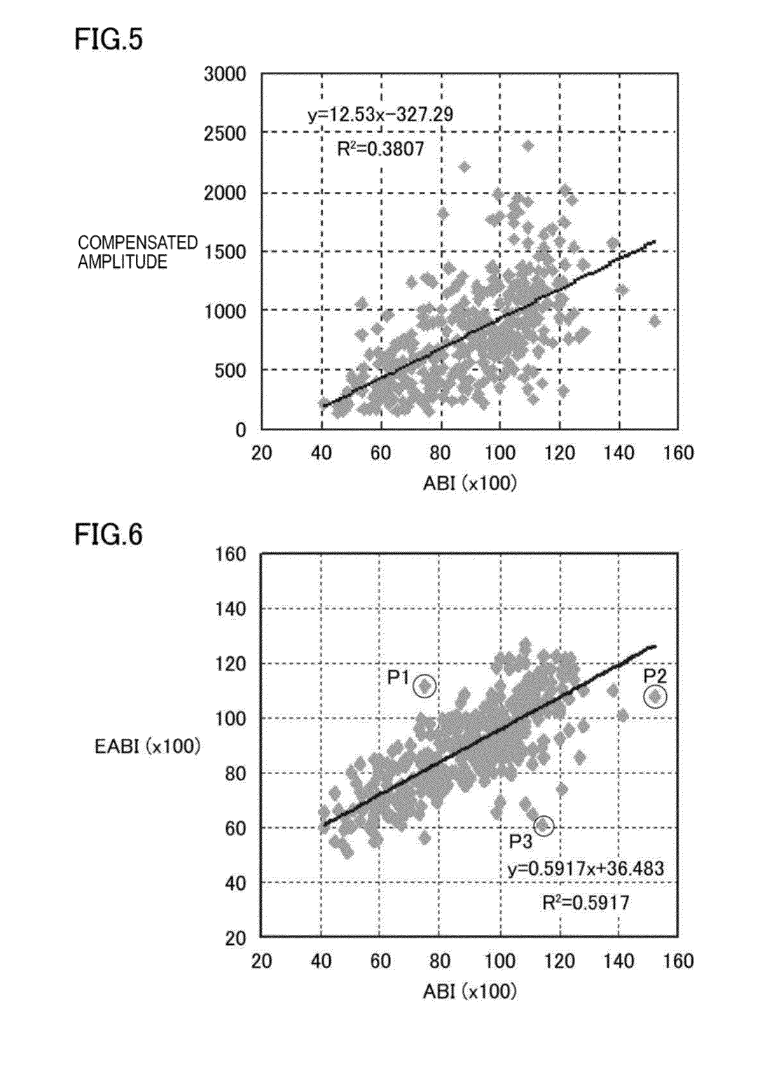

The inventors of the present application examined a correlation between these indices and the ABI. FIGS. 3 to 5 are graphs showing correlations between the ABI and % MAP, UT, and a pulse amplitude, respectively. These values were obtained by measuring the blood pressures and pulse waves of 200 adult males and females to calculate their ABIs and % MAP, UT, and pulse amplitude.

FIGS. 3 to 5 verify that a certain degree of correlation exists between the ABI and any of % MAP, UT, and pulse amplitude, respectively. Therefore, it is thought that any of % MAP, UT, and pulse amplitude may be used as the index of arteriostenosis corresponding to ABI. Alternatively, it is also thought that a combination of at least two of % MAP, UT, and pulse amplitude can be used as the index of arteriostenosis corresponding to ABI in order to enhance the correlation.

As an example, the inventors of the present application calculated a value by multiplying each of % MAP (A), UT (B), and pulse amplitude (C) by a conversion factor, as an index (the EABI), and examined the correlation between this index and the ABI. That is, the index was calculated according to the formula, EABI=aA+bB+cC+d (where a to d are coefficients), so as to compare this index with the ABI. FIG. 6 is a graph showing a correlation between the ABI and the EABI.

FIG. 6 verifies that a certain degree of correlation exists between the ABI and the index EABI calculated by combining % MAP (A), UT (B), and pulse amplitude (C), and FIG. 6 also verifies that this correlation is stronger than the correlation between the ABI and any one of % MAP, UT, and pulse amplitude.

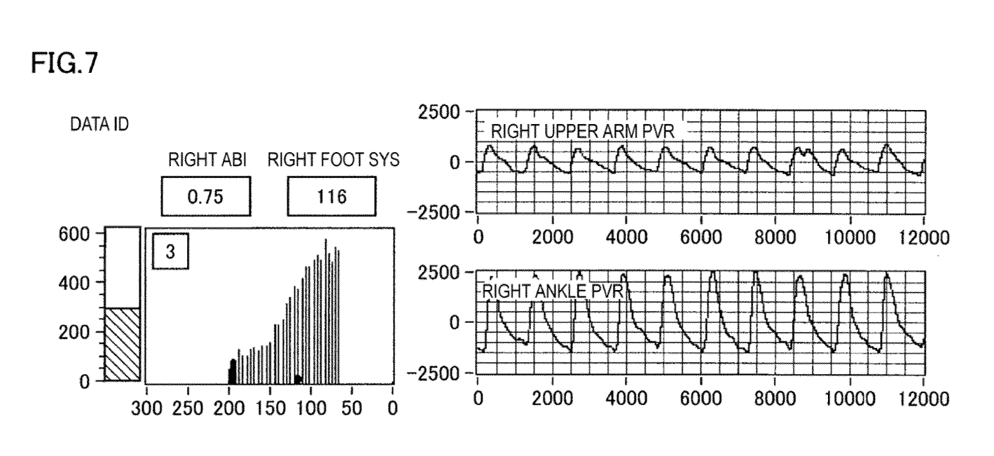

As indicated by P1 to P3 in FIG. 6, however, there are several measurements that significantly deviate from the regression line. FIGS. 7 to 9 are diagrams showing detailed measurement results of the subjects from whom the measurements denoted by P1-P3 were taken. Each of FIGS. 7 to 9 shows the ABI calculated from the blood pressure values of the right upper arm and the right ankle (right ABI), the systolic pressure value obtained from the blood pressure value of the right ankle, and sphygmograms taken from the right upper arm and the right ankle of the respective subjects. Additionally, FIGS. 7 to 9 each include a time-variation graph showing the pulse wave amplitude measured over time.

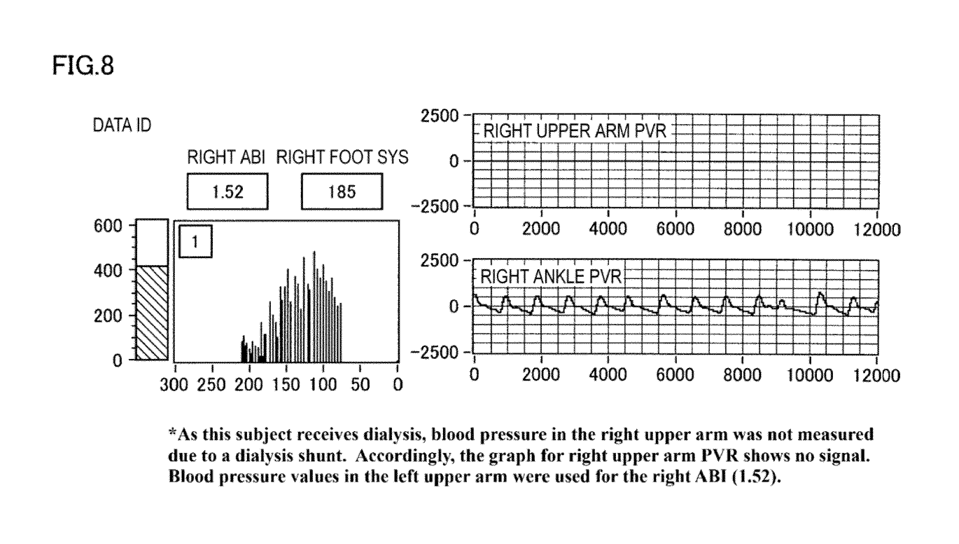

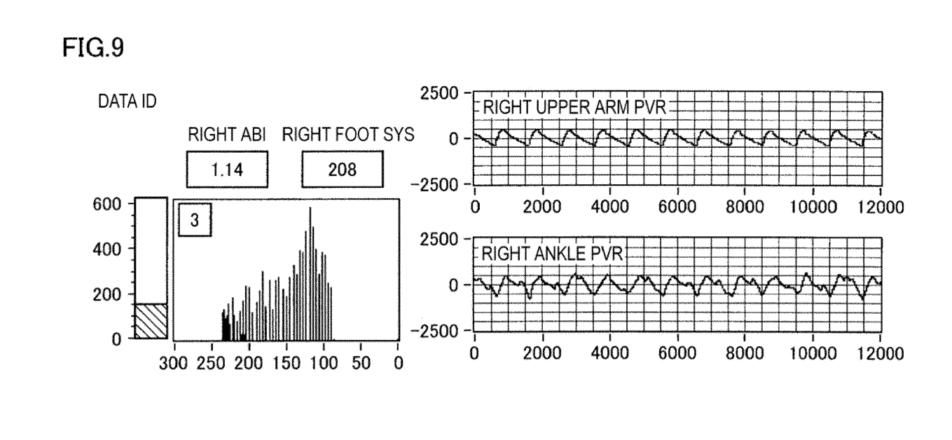

In the example of FIG. 7, the time-variation graph of the pulse wave amplitude is incomplete, and therefore there is the possibility that the blood pressure in the right ankle was not accurately measured. Moreover, in the examples shown in FIGS. 8 and 9, the time-variation graphs of the pulse wave amplitude show erratic or uneven measurement patterns, and therefore there is the possibility that a blood pressure in the right ankle was not accurately measured.

Based on the foregoing observation, those measurement values that greatly deviate from the regression line may be attributable to inaccurate pressure measurement. For this reason, the correlation is likely to be even stronger if these cases are excluded. That is, it is proved that one or more of % MAP, UT, and pulse amplitude can be used as the index of arteriostenosis corresponding to ABI.

Another example of the index of arteriostenosis corresponding to ABI may include a function for transfer of a pulse wave from the upper limb to the lower limb (a lower limb-upper limb pulse wave transfer function). This is because, in a transfer function in which a pulse wave in the upper limb is the input to the system (the vascular paths) and a pulse wave in the lower limb is the output from the system, changes may be found in a step response if angiostenosis exists in the system. That is, it is thought that this step response can be used as the index of arteriostenosis corresponding to ABI.

To verify this, the inventors of the present application measured the pulse waves of a healthy subject and a patient with arteriosclerosis obliterans (ASO) and calculated their step responses.

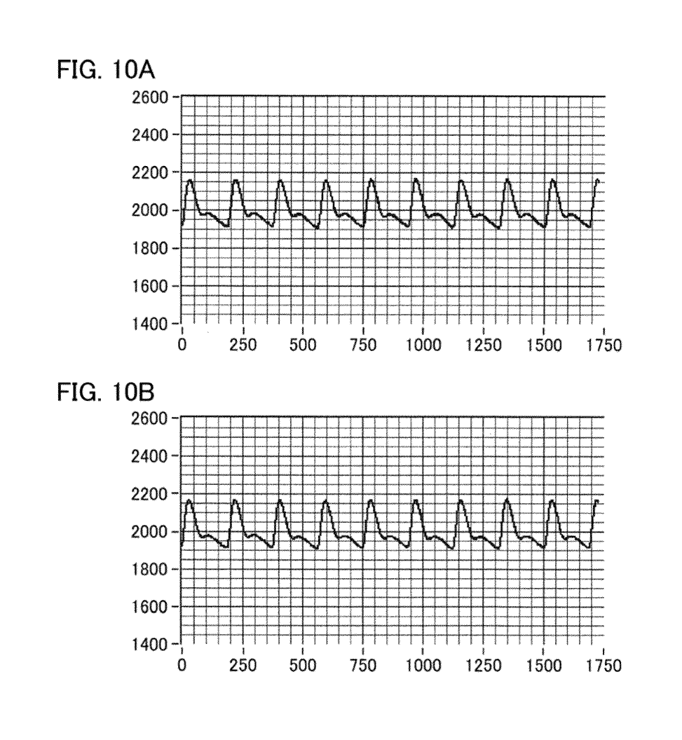

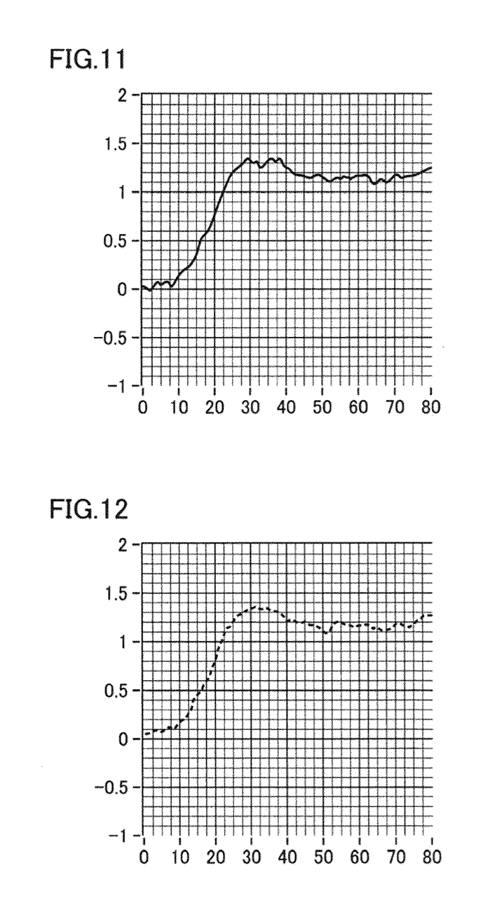

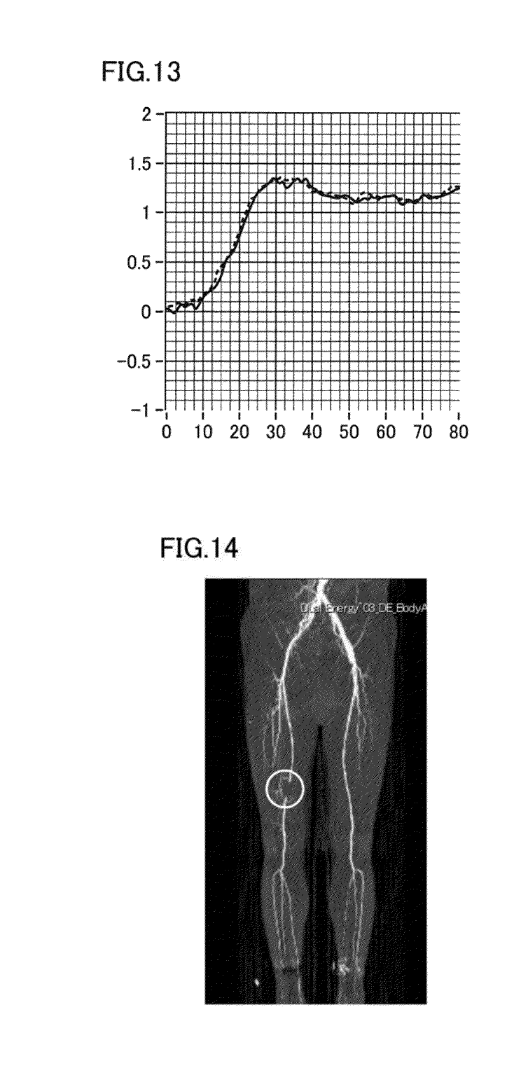

FIG. 10 shows the measurement results of pulse waves from the right ankle (A) and the left ankle (B) of the healthy subject. FIGS. 1 and 12 show the step response for the right upper arm to the right ankle (right step response) and the step response for the left upper arm to the left ankle (left step response) calculated from the measurement results in FIG. 10 and the pulse waves measured in the right and left upper arms, respectively. The comparison of these responses in FIG. 13 shows that they are nearly identical.

FIG. 14 is an X-ray image showing the arterial condition of the patient with arteriosclerosis obliterans. FIG. 14 shows an arterial occlusion in the circled region.

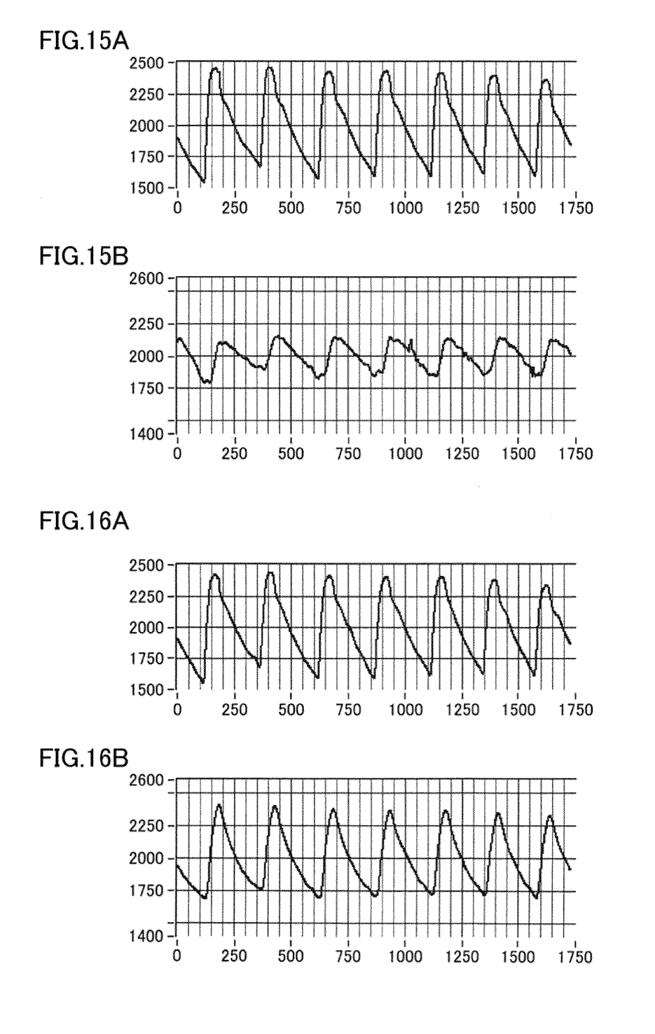

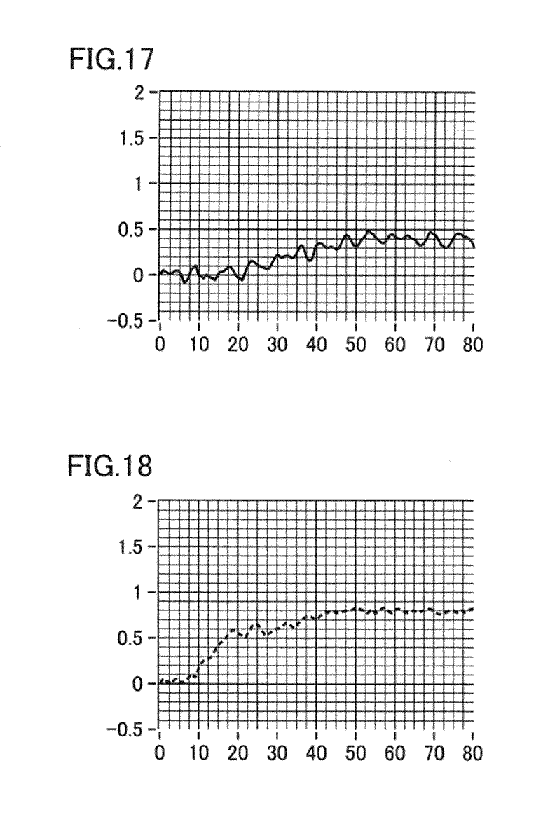

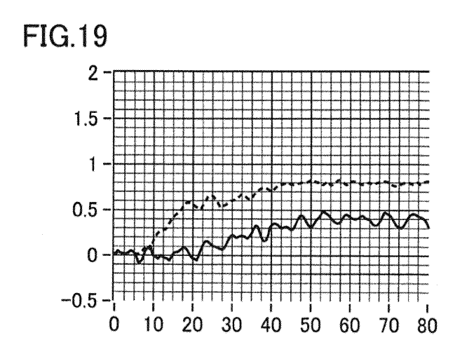

FIG. 15 shows the measurement results of the pulse waves in the right upper arm (A) and the right ankle (B) of the patient, and FIG. 16 shows the measurement results of the pulse waves in the left upper arm (A) and the left ankle (B) of the patient. FIGS. 17 and 18 show the right step response calculated from the pulse waves measured in the right upper arm and the right ankle in FIG. 15 and the left step response calculated from the pulse waves measured in the left upper arm and the left ankle in FIG. 16, respectively. As clearly seen in FIG. 19, the comparison of these responses show that to they are significantly different from each other.

That is, it can be said from this fact that less arterial occlusion exists as the correlation between the right and the left step responses is higher and arteriosclerosis is more likely to exist as the correlation is lower.

Accordingly, the inventors of the present application calculated degrees of arteriostenosis and variations in step responses using a circulatory system model. The circulatory system model employed by the inventors represents the vascular system of a body divided into multiple segments, One exemplary circulatory system model is the so-called “Avolio Model” described in Reference Literature 1. “Avolio, A. P., Multi-branched Model of Human Arterial System, 1980, Med. & Biol. Eng. & Comp., 18, 796”. The inventors used the Avolio Model as the circulatory system model for the calculations.

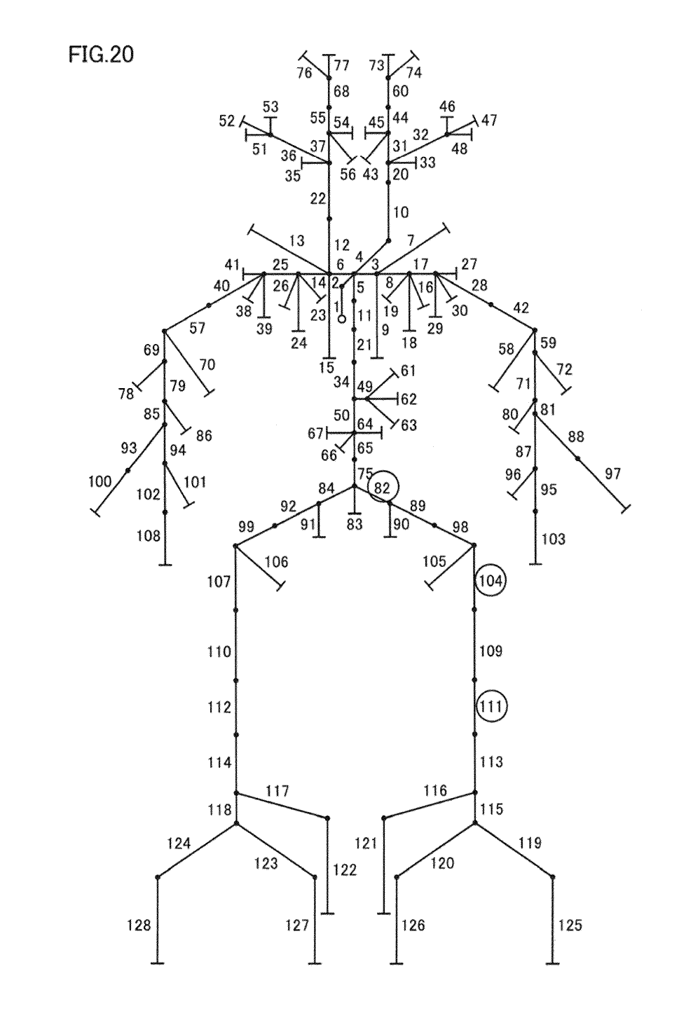

FIG. 20 is a Schematic Diagram of the Avolio Model.

With reference to FIG. 20, the Avolio Model divides the systemic arteries into 128 vascular elements (segments) and defines geometric values that represent the respective segments. In the Avolio model, the geometric values include a length, a radius, a vessel wall thickness, and a Young's modulus associated with the respective segments.

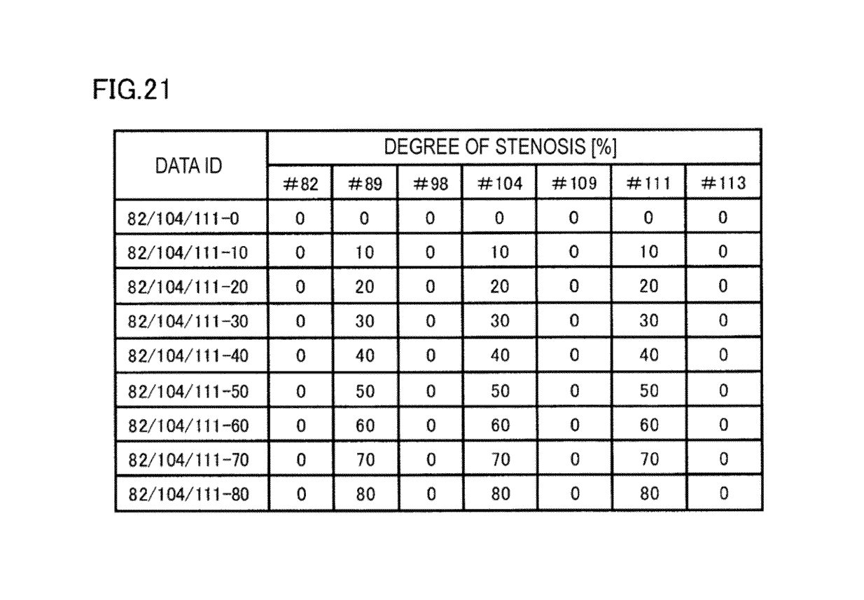

In the Avolio Model of FIG. 20, the inventors of the present application set parameters to create stenosis in the segments designated by the element numbers 82, 104, and 111 (circled in FIG. 20) in varying degrees and calculated the variations in the step responses. FIG. 21 is a table showing the degrees of stenosis created in the segments designated by the element numbers 82, 104, and 111 (circled in FIG. 20) in the Avolio Model that were used by the inventors to perform calculations. The degree of stenosis designated by Data ID “82/104/111-0” is set to zero percent for all of the segments so as to simulate or calculate the step response of the healthy subject. The larger the data ID number is, the greater the degree of stenosis in the segment is, thus producing a step response for the more advanced arteriosclerosis.

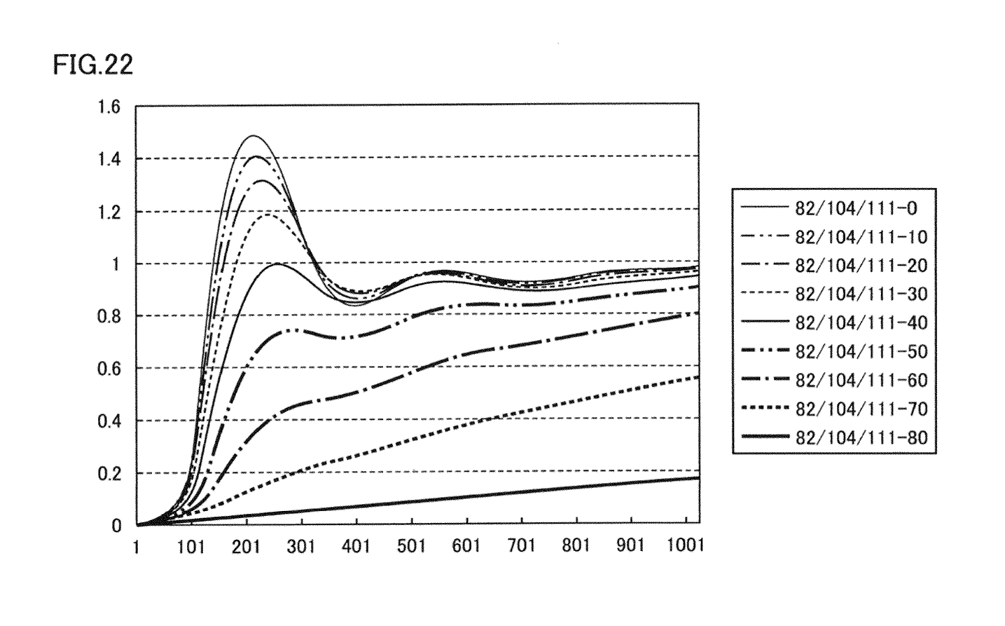

FIG. 22 is a graph plotting the results of the calculations. FIG. 22 shows the healthier the subject is, the steeper the rising is and the more rapidly the response drops after reaching the maximum, and that the greater the degree of stenosis in the subject, the gentler the rising is and the smaller the change in the response becomes after reaching the maximum.

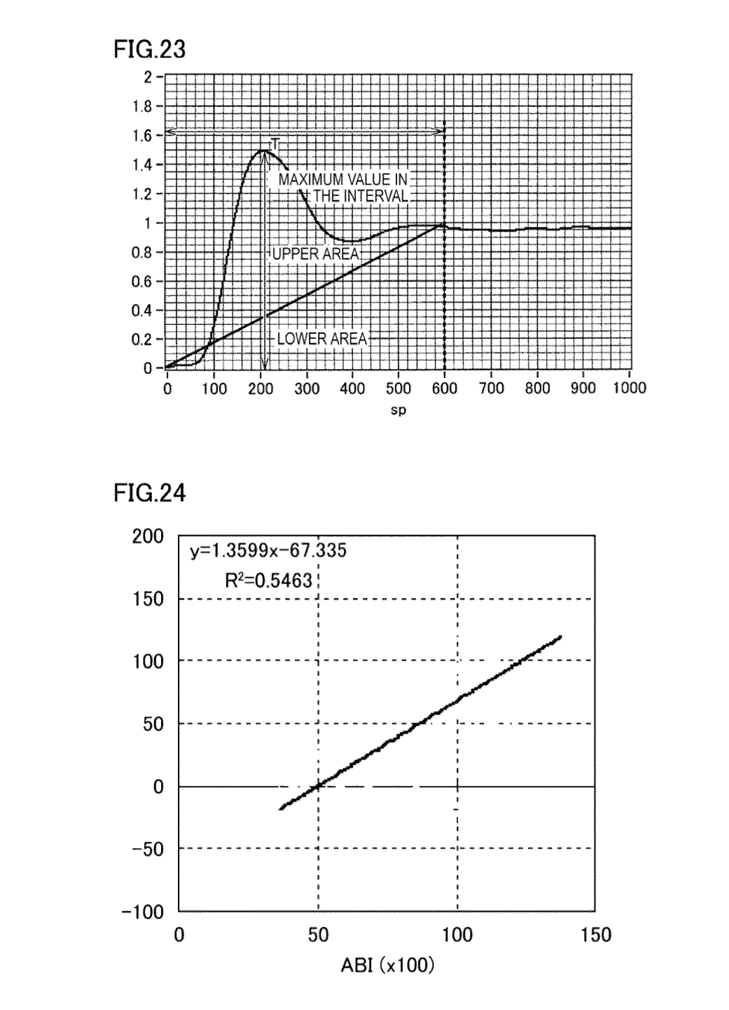

Therefore, as shown in FIG. 23, the inventors of the present application defined an upper area, the ratio of the upper area to the lower area, and the maximum value in the step response interval and examined whether or not these three values may be used as the index of arteriostenosis corresponding to ABI.

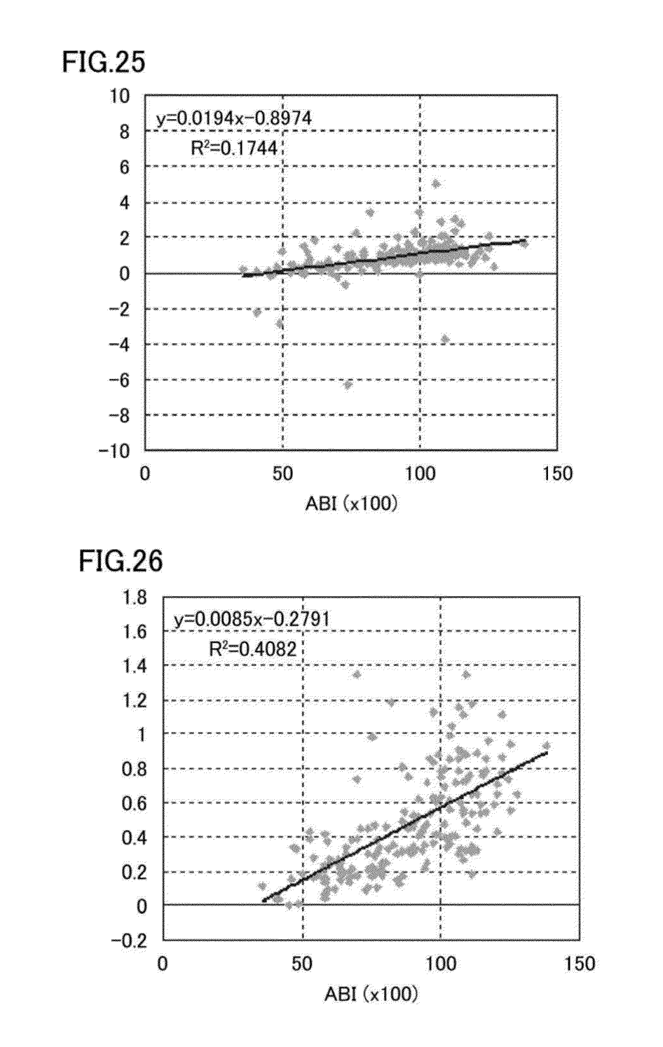

FIGS. 24 to 26 are graphs showing correlations between the ABI and the upper area, the ratio of the upper area to the lower area, and the maximum value in the interval, respectively. The measurements used for this purpose were the measurement results from the 200 adult males and females that were used to verify the correlations in FIGS. 3 to 5.

It was proved from FIGS. 24 to 26 that a certain degree of correlation exists between the ABI and any of the values and verify that a particularly strong correlation exists between the ABI and the upper area. Therefore, it is thought that the values obtained from the step response can be used as the index of arteriostenosis corresponding to ABI, and particularly the upper area calculated from the step response can be used as the index of arteriostenosis corresponding to ABI. Alternatively, it is also considered that a combination of at least two of the indices calculated from the % MAP, UT, pulse amplitude, and step response as described above can be used as the index of arteriostenosis corresponding to ABI in order to enhance the correlation.

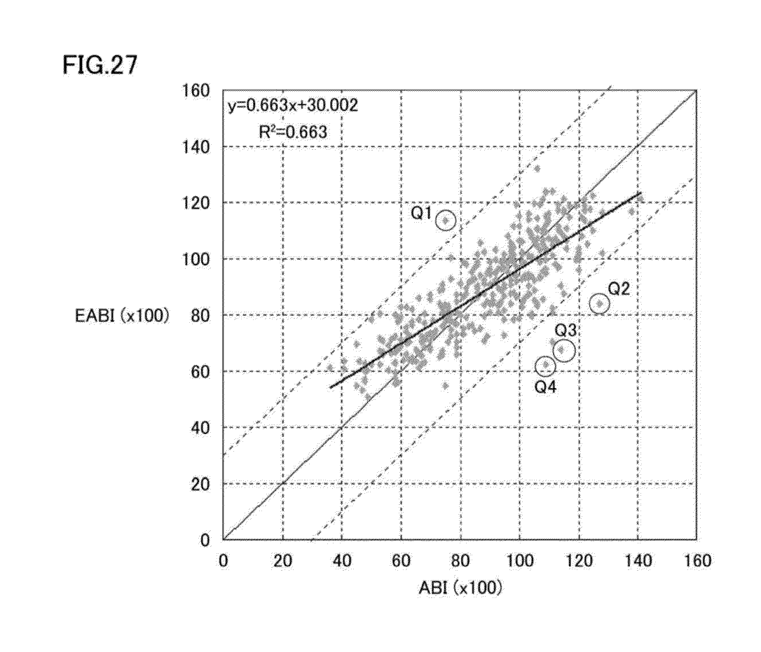

As an example, the inventors of the present application calculated, as an index, a value (EABI) by multiplying each of % MAP (A). UT (B), pulse amplitude (C), and the index calculated from the step response (D) (e.g., the upper area) by a conversion factor, and examined the correlation between this index and the ABI. That is, the index was calculated according to the formula, EABI=aA+bB+cC+dD+e (where a to e are coefficients), so as to compare this index with the ABL. FIG. 27 is a graph showing a correlation between the ABI and the EABI.

It was proved from FIG. 27 that the index EABI calculated by combining % MAP (A), UT (B), pulse amplitude (C), and the index calculated from the step response (D) (e.g., the upper area) had a considerably high correlation with the ABI, and that this correlation is stronger than the correlation between the ABI and any one of or a combination of % MAP, UT, and pulse amplitude.

As in the cases discussed in relation to FIG. 6, there are several measurements that significantly deviate from the regression line as indicated by Q1-Q4 in FIG. 27. As in FIG. 6, examination of these measurement results shows the blood pressure measurements are unreliable for all these cases. For this reason, the correlation is likely to be even stronger if these cases are excluded.

Operational Flow

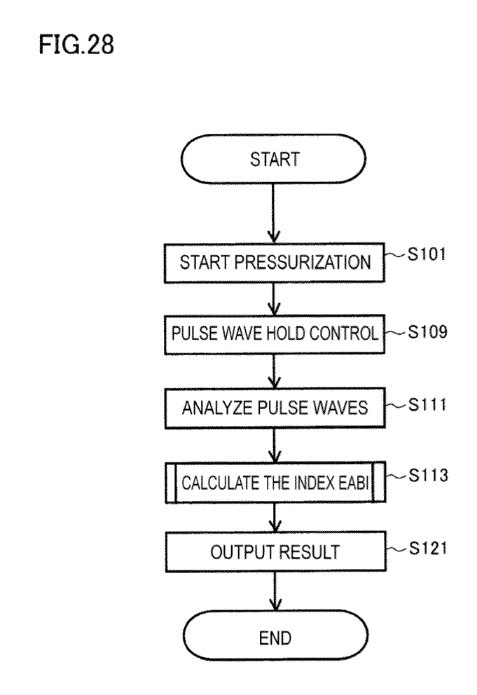

FIG. 28 is a flowchart representing a specific example of the operational flow that occurs in the measurement device 100. The operation represented in the flowchart of FIG. 28 is carried out on the CPU 10 as the CPU 10 reads out and executes a program stored in the ROM 12 while using the RAM 14 as the work memory so as to execute the functionalities shown in FIG. 2.

With reference to FIG. 28, the CPU 10 starts pressurization of the cuff 24 in Step S101, and keeps the pressurization until the pressure reaches a pressure suitable for the measurement of a pulse wave. Then, the CPU 10 performs a hold control for keeping the cuff pressure in the suitable pressure. This pressure corresponds to, for example, a constant pressure of approximately 50 to 60 mmHg or a pressure lower than the diastolic pressure value by 5 to 10 mmHg. In Step S111, the CPU 10 analyzes pulse waves obtained based on change in the cuff pressure during the hold control, and calculates the index EABI corresponding to the ABI as the index of arteriostenosis.

The CPU 10 outputs the index EABI of arteriostenosis calculated from the pulse waves in Step S121. This output may be displayed on a screen or transmitted to a separate device, such as a PC or an external recording medium.

It should be noted that examples of the calculating method in the Step S113 include various calculating methods. This is because, as described above, any one or a combination of two or more of % MAP, UT, pulse amplitude, and lower and a lower limb-upper limb pulse wave transfer function (e.g., the upper area) can be used as the index EABI.

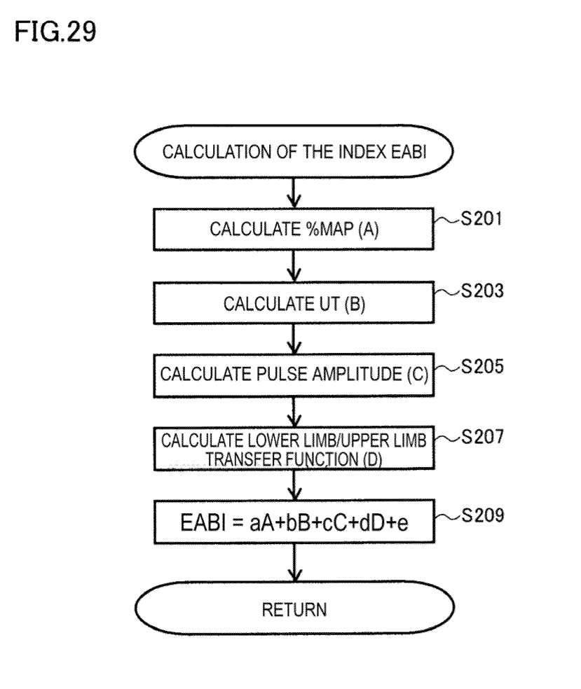

For example, FIG. 29 shows a flowchart representing a specific example of the operation in the foregoing Step S113 in which all of the above are combined for the calculation of the index EABI. As described above, the index EABI thus calculated has a high correlation with the ABI, and therefore can be used as the index of arteriostenosis with high accuracy.

With reference to FIG. 29, in Steps S201-207, the CPU 10 calculates % MAP (A), UT (B), pulse amplitude (C), and a lower limb-upper limb pulse wave transfer function (D) (e.g., the upper area) in order. Of course, this calculation order is not limited to the order shown in FIG. 29.

Subsequently, in Step S209, the CPU 10 calculates the index EABI=aA+bB+cC+dD+e (where a to e are coefficients) using a conversion factor defined in advance.

### Advantageous Effects of Embodiments

By performing the above operation, an index indicating the presence or absence of stenosis or the degree of stenosis in arteries corresponding to ABI can be calculated from the pulse waves.

As mentioned above, it is known that blood pressure values are susceptible to calcification of the arteries. Also, the subject may have unstable pulse amplitude due to arrhythmia or small pulse amplitude due to angiostenosis and it is also known that blood pressure values are susceptible to these conditions.

In contrast, since the wave pulses are calculated based on waveforms for several heartbeats, it is less susceptible to the aforementioned conditions. Therefore, the case where the index is calculated from a pulse wave is harder to be affected by arrhythmia or calcification than the conventional case where the index is calculated from a blood pressure value, so that the index is accurately calculated in the former case.

Although it is possible to use any one of the indices (% MAP, UT, pulse amplitude, and the lower limb-upper limb pulse wave transfer function (e.g. the upper area)) obtained from the pulse waves as an index indicating the presence or absence of stenosis or the degree of stenosis in arteries, the more accurate index can be obtained by combining these indices. Furthermore, the inventors of the present application proved that it is possible to obtain a particularly accurate index by using or combining the lower limb-upper limb pulse wave transfer function (e.g., the upper area) in particular.

Moreover, since there is no need to measure a blood pressure, it is possible to perform a measurement concerning a subject not in a supine position but in a sitting position, and to remarkably enhance simplicity as a screening test.

Furthermore, a program for causing the measurement device 100 or an arithmetic device such as a personal computer (upon obtaining values/data from the measurement device 100) to calculate the above index indicating the presence or absence of stenosis or the degree of stenosis in arteries from the pulse waves may also be provided. Such a program may be provided as a program product by storing the program on a computer-readable recording medium, such as a flexible disk, a CD-ROM (compact disk-read only memory), a ROM (read only memory), a RAM (random access memory), and a memory card associated with a computer. Also, such a program can be recorded on a computer-readable recording medium included in a computer, such as a hard disk, and provided as a program product. Moreover, the program may be provided by allowing it to be downloaded via a network.

It should be noted that the program according to the present invention may invoke necessary modules, among program modules provided as part of a computer operating system (OS), in a predetermined sequence at predetermined timings, and cause such modules to perform processing. In this case, processing is executed in cooperation with the OS, without the above modules being included in the program itself. Such a program that does not include such modules can also be the program according to the present invention.

Also, the program according to the present invention may be provided incorporated in part of another program. In this case as well, processing is executed in cooperation with the other program, with the modules of the other program not included in the program itself. Such a program incorporated in another program can also be the program according to the present invention.

The program product that is provided is executed after being installed in a program storage unit such as a hard disk. Note that the program product includes the program itself and the recording medium on which the program is stored.

The embodiments of the present invention described above are to be considered in all respects only to be illustrative and not restrictive. The scope of the invention is indicated by the appended claims rather than the above description, and all changes which come within the meaning and range of equivalency of the claims are to be encompassed within the scope of the invention.

### REFERENCE SIGNS LIST

1 Information processing unit
2 Control unit
4 Output unit
6 Operation unit
8 Storage device
12 ROM
14 RAM
20, 20al, 20ar, 20bl, 20br Detection unit
22al, 22ar, 22b1, 22br, 27al, 27ar, 27bl, 27br Tube
24, 24al, 24ar, 24bl, 24br Cuff
25, 25al, 25ar, 25bi, 25br Pressure pump
26, 26al, 26ar, 26bl, 26br Pressure regulating valve
28, 28al, 28ar, 28bl, 28br Pressure sensor
29, 29al, 29ar, 29bl, 29br Converter
30 Adjustment unit
100 Measurement device
102 Pulse wave measurement unit
104 Calculation unit

1. A measurement device for measuring a pulse wave and calculating an index of arteriostenosis from the pulse wave, comprising:
a measurement unit to be mounted on a measurement site configured to measure a value corresponding to a load given to the measurement site; and
an arithmetic device connected to the measurement unit,
wherein the arithmetic device comprises
a pulse wave measurement unit configured to measure a pulse wave based on a measurement value in the measurement unit,
a first calculation unit configured to measure a predetermined parameter value from the pulse wave, and
a second calculation unit configured to calculate an index value corresponding to Ankle Brachial Blood Pressure Index (ABI) as the index of arteriostenosis using the parameter value calculated by the first calculation unit, and
the first calculation unit calculates, as the predetermined parameter value, from the pulse wave, at least one of a normalized pulse wave area (% MAP), which is an index indicating a sharpness of the pulse wave, an upstroke time (UT), which is an index indicating a rising feature value of an ankle pulse wave, a pulse amplitude, and an index value indicating a lower limb-upper limb pulse wave transfer function, which is a function for transfer of a pulse wave from the upper limb to the lower limb.

2. The measurement device according to claim 1, wherein the measurement unit comprises a cuff for being mounted on the measurement site and a sensor for detecting a pressure inside the cuff,
the arithmetic device is connected to the sensor, and
the pulse wave measurement unit measures a pulse wave from the sensor.

3. (canceled)

4. The measurement device according to claim 1, wherein the second calculation unit calculates the index value by combining two or more of the % MAP, UT, pulse amplitude, and index value indicating a lower limb-upper limb pulse wave transfer function that are calculated by the first calculation unit.

5. The measurement device according to claim 1, wherein the second calculation unit calculates the index value by combining the index value indicating a lower limb-upper limb pulse wave transfer function and at least one of the % MAP, UT, and pulse amplitude that are calculated by the first calculation unit.

6. An index calculating method for calculating an index value of arteriostenosis from a pulse wave, the calculating method comprising the steps of:
obtaining the pulse wave;
calculating a predetermined parameter value from the pulse wave; and
calculating an index value corresponding to Ankle Brachial Blood Pressure Index (ABI) as the index of arteriostenosis using the parameter value,
wherein the step of calculating a predetermined parameter value calculates, as the predetermined parameter value, from the pulse wave, at least one of a normalized pulse wave area (% MAP), which is an index indicating a sharpness of the pulse wave, an upstroke time (UT), which is an index indicating a rising feature value of an ankle pulse wave, a pulse amplitude, and an index value indicating a lower limb-upper limb pulse wave transfer function, which is a function for transfer of a pulse wave from the upper limb to the lower limb.

7. A non-transitory computer-readable storage medium storing an index calculating program for causing a computer to execute processing for calculating an index value of arteriostenosis from a pulse wave, the program fer-causing the computer to execute the steps of:
obtaining the pulse wave;
calculating a predetermined parameter value from the pulse wave; and
calculating an index value corresponding to Ankle Brachial Blood Pressure Index (ABI) as the index of arteriostenosis using the parameter value,
wherein the step of calculating a predetermined parameter value causes the computer to execute processing for calculating, as the predetermined parameter value, from the pulse wave, at least one of a normalized pulse wave area (% MAP), which is an index indicating a sharpness of the pulse wave, an upstroke time (UT), which is an index indicating a rising feature value of an ankle pulse wave, a pulse amplitude, and an index value indicating a lower limb-upper limb pulse wave transfer function, which is a function for transfer of a pulse wave from the upper limb to the lower limb.
